(19) United States
(12) Patent Application Publication
(10) Pub. No.: US 2024/0189497 A1
(43) Pub. Date: Jun. 13, 2024

(54) NASAL SUCTION DEVICE WITH MODIFIED HANDLE

(71) Applicant: TAI-SHINY TECHNOLOGY CO., LTD., New Taipei City (TW)

(72) Inventor: Chang-Chi LEE, New Taipei City (TW)

(21) Appl. No.: 18/528,518

(22) Filed: Dec. 4, 2023

(30) Foreign Application Priority Data
Dec. 7, 2022 (TW) 111213468

(51) Int. Cl. A61M 1/00

(52) U.S. Cl. CPC A61M 1/76; A61M 1/81

(57) ABSTRACT

A nasal suction device with a modified handle includes: an extraction structure; a suction structure having a suction head with a suction hole; an air extraction structure including a pump with an air inlet, a piston movably installed in the pump, a handle, a first check valve corresponding to the air inlet, and a second check valve installed to the pump or piston; and a connecting tube with two ends connected to the suction hole and the air inlet respectively. The handle includes a rod connected to the piston, and an operating handle formed outside the pump and including a lever and a connecting section. A finger hole is formed and enclosed by the lever and the inner side of the connecting section, and a finger slot is formed by the lever and the outer side of the connecting section.

### TECHNICAL FIELD

The present disclosure relates to a nasal suction device, and more particularly relates to a nasal suction device with a modified handle.

### RELATED ART

It is a common physiological reaction for the nasal cavity to be filled with nasal mucus in order to protect the respiratory tract, in which running nose may also be caused by colds and flu and the nasal mucus contains germs and white blood cells killed by immune responses. However, allergies, illnesses, and other conditions can cause the nasal mucosa to swell and result in nasal congestion. For newborn babies, since they can only breathe through their nose, nasal congestion may cause asphyxia, and for children, the accumulation of nasal mucus in the nasal cavity over a long period of time may lead to sinusitis or other chronic diseases, and they will suffer from nasal congestion for the rest of their lives.

Based on the above reasons, the present applicant has developed a nasal suction device as disclosed in R.O.C. Pat. No. TWM574495 with the advantages of having a simple structure, requiring no electricity, operating with ease, improving the safety of use, etc. and this patented device is widely used by consumers. However, the handle of this nasal suction device will cause fatigue, soreness and discomfort of the middle finger and ring finger when used for a long time.

In view of the above drawbacks, the present applicant based on years of experience to conduct extensive research and experiment, and finally provided a nasal suction device with a modified handle of this disclosure to improve the drawbacks of the prior art.

### SUMMARY OF THE DISCLOSURE

The objective of the present disclosure is to provide a nasal suction device with a modified handle, in which forces are uniformly exerted onto the handle, so that it is easy to apply force for the operation and does not cause discomfort of the fingers when used for a long time.

To achieve the aforementioned objective, the present disclosure provides a nasal suction device with a modified handle including a suction structure, an air extraction structure and a connecting tube. The suction structure includes a suction head with a suction hole. The air extraction structure includes a pump, a piston, a handle, a first check valve and a second check valve. The pump has an air inlet, and the handle includes a rod and an operating handle connected to the rod, where an end of the rod away from the operating handle is connected to the piston, the piston is movably installed in the pump, the operating handle is formed outside the pump. The first check valve is disposed corresponding to the air inlet, and the second check valve is installed at one of the pump and the piston. The connecting tube has two ends connected to the suction hole and the air inlet respectively. The operating handle includes a lever and two connecting sections extended from the lever and connected to the rod, a finger hole is formed and enclosed by the lever and inner side of each of the connecting sections, and two finger slots are formed at the lever and on an outer side of each connecting section.

The present disclosure further has the following effects. With the configuration of the restoring spring, the present disclosure greatly reduces the frequency of operation of each finger to improve the performance of use. The separation between the finger hole and each finger slot facilitates users to pass and position each finger. The curved shape of the lever and each connecting section provides excellent structural strength.

### BRIEF DESCRIPTION OF THE DRAWINGS

FIG. 1 is a perspective view of a nasal suction device with a modified handle in accordance with the present disclosure:

FIG. 2 is an exploded view of a nasal suction device with a modified handle in accordance with the present disclosure:

FIG. 3 is a first cross-sectional view showing a using status of a nasal suction device with a modified handle in accordance with the present disclosure:

FIG. 4 is a second cross-sectional view showing a using status of a nasal suction device with a modified handle in accordance with the present disclosure; and

FIG. 5 is a cross-sectional view showing a using status in accordance with another embodiment of the present disclosure.

### DETAILED DESCRIPTION

The technical characteristics of this disclosure will become apparent with the detailed description of preferred embodiments accompanied with the illustration of related drawings as follows. It is noteworthy that the embodiments and drawings disclosed herein are to be considered illustrative rather than restrictive.

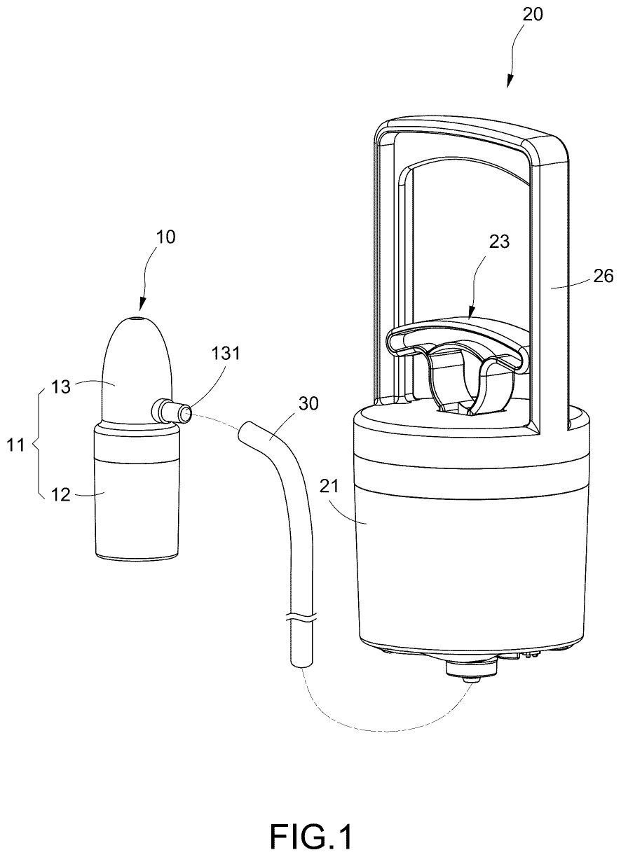

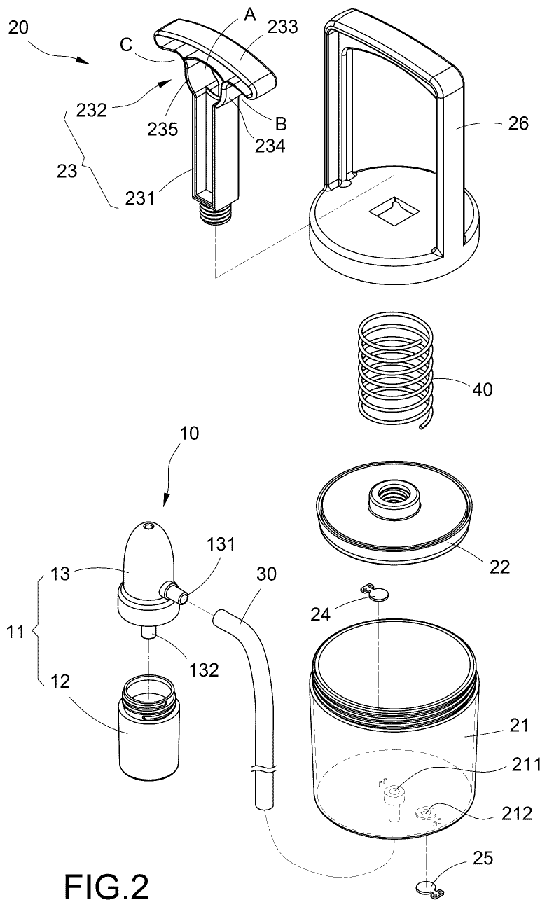

With reference to FIGS. 1 to 4 for a nasal suction device with a modified handle in accordance with the present disclosure, the nasal suction device includes a suction structure 10, an air extraction structure 20 and a connecting tube 30.

The suction structure 10 includes a suction head 11, and the suction head 11 includes a storage barrel 12 and a cover 13, a suction hole 131 is formed at a side edge of the cover 13, and a drawing tube 132 is installed in the cover 13. After the cover 13 is assembled with the storage barrel 12, the drawing tube 132 and the suction hole 131 communicate to each other.

The air extraction structure 20 includes a pump 21, a piston 22, a handle 23, a first check valve 24 and a second check valve 25. The pump 21 is a cylinder with two closed ends, an air inlet 211 is formed at the middle position of the bottom of the pump 21, and a through hole 212 is formed at a position with an appropriate distance from the air inlet 211. The handle 23 includes a rod 231 and an operating handle 232 connected to the rod 231, an end of the rod 231 away from the operating handle 232 is connected to the piston 22, the piston 22 is movably installed in the pump 21, the operating handle 232 is formed outside the pump 21, the first check valve 24 is disposed corresponding to the air inlet 211, and the second check valve 25 of this embodiment is installed onto the through hole 212 of the pump 21.

The operating handle 232 includes a lever 233 and two connecting sections 234, 235 respectively and downwardly extended from the lever 233 and connected to the rod 231, a finger hole A is formed and enclosed by the lever 233 and an inner side of each connecting section 234, 235, and two finger slots B, C are formed at the lever 233 and on an outer side of each connecting section 234, 235.

In an embodiment, the lever 233 is an arc rod, each connecting section 234, 235 is symmetrically and downwardly extended along the plumb line from the center of the lever 233 and jointly connected to the rod 231, each connecting section 234, 235 is formed by connecting two different arc radii, and the centers of the arc radii are disposed on two different sides of each connecting section 234, 235. Each arc radius adjacent to the rod 231 has the effect of enhancing the overall structure, and each arc radius away from the rod 231 is conducive to the comfort of each finger after it is inserted. The finger hole A is a closed through hole, and each finger slot B, C is an open through slot.

In an embodiment, the air extraction structure 20 further includes a lift handle 26 extended upwardly from the pump 21 and formed above the handle 23. In addition to the easy carrying and transporting effects, the lift handle 26 further provides a support for human palm, so as to facilitate users to pull and lift the handle 23.

The connecting tube 30 is a soft tube made of plastic or silicone, and two ends of the connecting tube 30 are connected to the suction hole 131 and the air inlet 211 respectively. The soft connecting tube 30 not only facilitates displacing the suction structure 10, but also facilitates winding the connecting tube 30 or folding and stacking the suction structure 10 and the air extraction structure 20 with each other in order to save storage space.

In an embodiment of the present disclosure, the nasal suction device with a modified handle further includes a restoring spring 40 installed in the pump 21 and disposed between the piston 22 and an end wall of the pump 21. The elastic restoring force of the restoring spring 40 enables the piston 22 and the handle 23 to move together toward the bottom of the pump 21 and restore their original positions. In this embodiment, the restoring spring 40 sheathes the outer periphery of the rod 231.

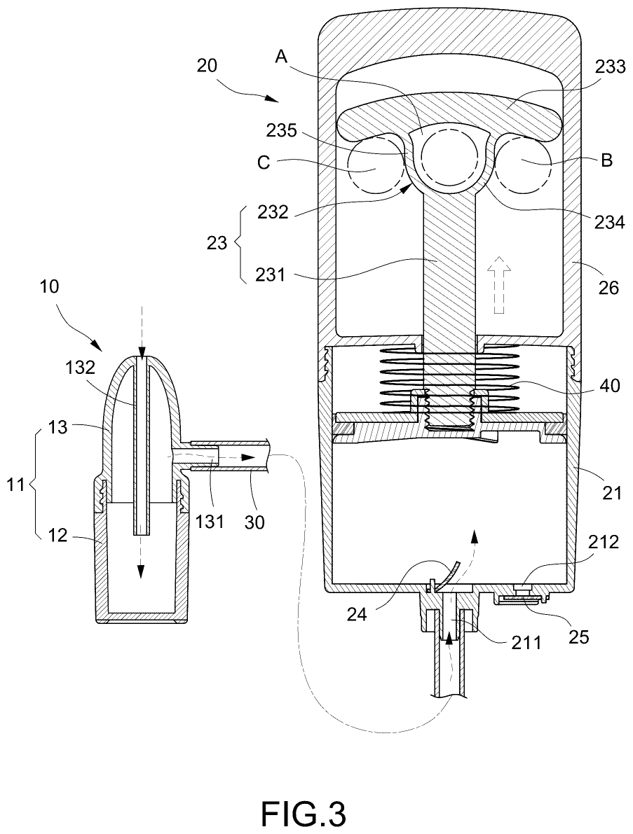

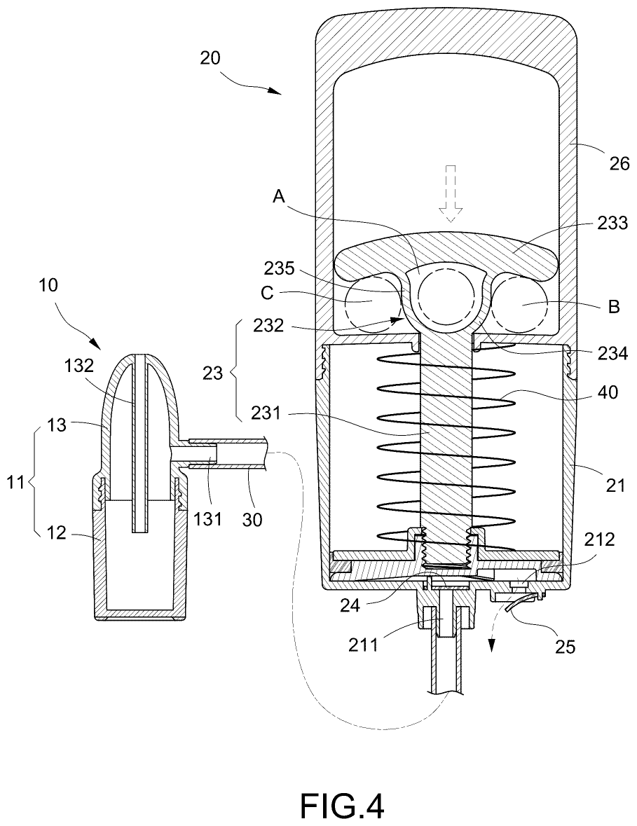

When use, the cover 13 of the suction head 11 is placed in the user's nostrils (not shown in the figure), and the drawing tube 132 is placed at a position corresponding to the user's nostrils. After each finger passes into the finger hole A and each finger slot B, C, the lift handle 26 is used as a fulcrum to apply force, such that when the piston 22 is continuously (or intermittently) driven in the pump 21 for reciprocal action by means of a manual operation in conjunction with the operation of the restoring spring 40, and the first check valve 24 and the second check valve 25 cooperate to produce a suction and an exhaust action in the pump 21. During the suction (as shown in FIG. 3), the piston 22 is pulled upward to drive the suction structure 10 to draw external air into the pump 21 through the connecting tube 30 and let the air entering into the pump 21 push the first check valve 24 to an open state (at this time, the second check valve 25 is in a closed state), resulting in the formation of a negative pressure in the pump 21 to generate the suction. During the exhaust action (as shown in FIG. 4), the restoring spring 40 is provided to drive the piston 22, the air in the pump 21 pushes the first check valve 24 to the closed state, and the air may not be discharged from the connecting tube 30 to the suction structure 10. At the same time, the air in the pump 21 pushes the second check valve 25 to the open state, which generates the exhaust action in the pump 21. In this way, the piston 22 in the pump 21 is driven to let the suction structure 10 generate a suction force through the connecting tube 30, suck the nasal mucus by the drawing tube 132 of the cover 13, and draw the nasal mucus from the drawing tube 132 to the storage barrel 12.

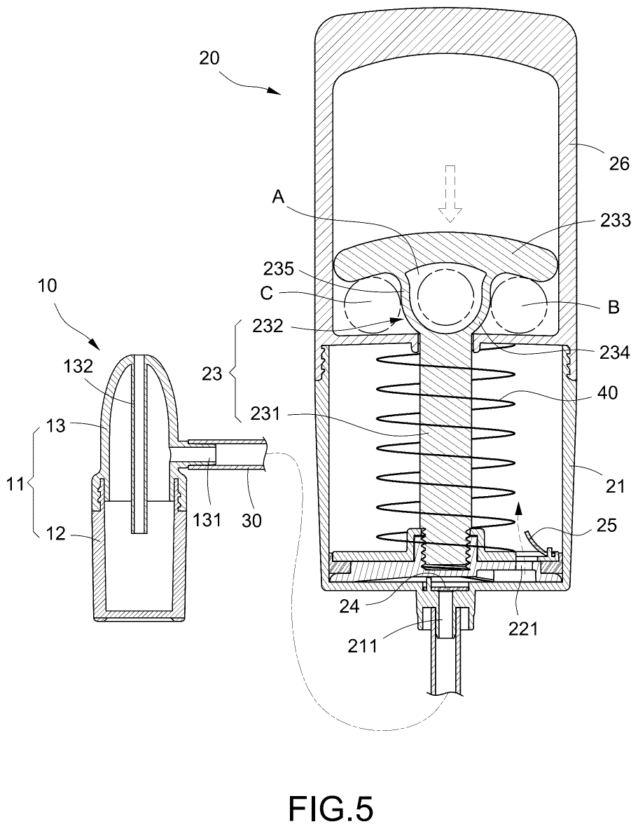

With reference to FIG. 5 for a nasal suction device with a modified handle in accordance with another embodiment of the present disclosure, this embodiment is substantially the same as the previous embodiment, except this embodiment further includes a through hole 221 formed at the piston 22. During the suction, the first check valve 24 is in the open state, the second check valve 25 is in the closed state, and a negative pressure is formed in the pump 21 to generate the suction. During the exhaust action, the restoring spring 40 drives the piston 22 to let the air in the pump 21 push the first check valve 24 to a closed state, and the air may not be discharged from the connecting tube 30 to the suction structure 10. At the same time, the air below the piston 22 pushes the second check valve 25 to the open state, and the air entering above the piston 22 to generate the exhaust action, so as to achieve the same effect of the previous embodiment.

In summation of the description above, the present disclosure achieves the intended purpose of use, overcomes the drawbacks of the related art.

What is claimed is:

1. A nasal suction device, comprising:
a suction structure, comprising a suction head, the suction head comprising a suction hole;
an air extraction structure, comprising a pump, a piston, a handle, a first check valve and a second check valve, the pump comprising an air inlet, the handle comprising a rod and an operating handle coupled to the rod, wherein an end of the rod away from the operating handle is coupled to the piston, the piston is movably installed in the pump, the operating handle is disposed outside the pump, the first check valve is disposed corresponding to the air inlet, and the second check valve is installed on one of the pump and the piston; and
a connecting tube, coupled to the suction hole and the air inlet respectively by two ends thereof;
wherein, the operating handle comprises a lever and two connecting sections respectively extended from the lever and coupled to the rod, a finger hole is defined and enclosed by the lever and inner side of each of the connecting sections, and two finger slots are defined by the lever and outer side of each of the connecting sections.

2. The nasal suction device according to claim 1, further comprising a restoring spring installed in the pump and disposed between the piston and an end wall of the pump.

3. The nasal suction device according to claim 2, wherein the restoring spring is adapted to sheathe outer periphery of the rod.

4. The nasal suction device according to claim 1, wherein the lever is an arc-shaped rod, and the connecting sections are symmetrically and downwardly extended along a plumb line from a center of the lever and jointly coupled to the rod.

5. The nasal suction device according to claim 4, wherein each connecting section is structured with two arc radii in different, and centers of the arc radii are located on two sides of each connecting section.

6. The nasal suction device according to claim 1, wherein the finger hole is a closed through hole.

7. The nasal suction device according to claim 1, wherein the finger slot is an open through slot.

8. The nasal suction device according to claim 1, wherein the air extraction structure further comprises a lift handle extended from the pump and disposed above the handle.
